(19) United States
(12) Patent Application Publication
(10) Pub. No.: US 2021/0145458 A1
(43) Pub. Date: May 20, 2021

(54) THREE-DIMENSIONAL ORIENTATION SYSTEM AND METHOD FOR ORTHOPEDIC SURGERY

(71) Applicants: Thomas Paszicsnyek, Bruck/Mur (AT); Bernardo Innocenti, Brussels (BE)

(72) Inventors: Thomas Paszicsnyek, Bruck/Mur (AT); Bernardo Innocenti, Brussels (BE)

(21) Appl. No.: 16/904,823

(22) Filed: Jun. 18, 2020

Related U.S. Application Data
(60) Provisional application No. 62/937,524, filed on Nov. 19, 2019.

(51) Int. Cl. A61B 17/15; A61B 34/10; A61B 34/00

(52) U.S. Cl. CPC A61B 17/154; A61B 2017/00991; A61B 34/25; A61B 34/10

(57) ABSTRACT

A system for orthopedic surgery is provided that includes a client device; a plurality of position sensor units configured to communicate position information wirelessly to the client device, where each of the position sensor units includes an anchoring means for attaching position sensor units to bone; and an adjustable cutting block. The cutting block preferably includes an attachment portion having a recess therein for attaching the cutting block to a first of the plurality of sensor units; a cutting block portion having a second recess for attaching the cutting block to a second of the plurality of sensor units and an aperture extending through the cutting block portion for guiding a bone cutting instrument; and an intermediate portion coupling the attachment portion and the cutting block portion to each other, the cutting block therewith configured to adjustably set an orientation of the cutting instrument.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE INVENTION

### Field of the Invention

This application generally relates to prosthetic implants, and in particular, systems and methods for determining correct implant positioning in, for example, joint replacement procedures using individual kinematics and/or muscle activity measurements.

### Description of the Related Art

Due to increasing numbers of joint replacements and consecutive higher number of dissatisfied patients, there is an increased demand for implant positioning that accounts for individual performance and situation. For example, knee prosthetic joint implants and procedures for total knee arthroplasty (“TKA”) continue to change. This increase coupled with the desire of patients to maintain an active lifestyle post implant has led to increased demand for artificial joints to perform closer to normal functions.

The design of knee prosthetic implants has seen considerable change since its inception based primarily on a better understanding of the motion and stability of the natural knee. Knee prosthetic implants ideally seek to match the kinematic and stability behavior of a healthy living knee. However, existing systems account only for the anatomical situation of the bones, neglecting the soft tissue envelope, its function, and muscular abilities related to the individual conditions of the patient. Additionally, existing systems rely on qualitative assessments by surgeons pre- and post-operatively, which may vary depending on the experience of the practitioner. Accordingly, there is a need to account for such factors to ensure implantation of artificial joints that perform closer to healthy joints and/or use quantitative information to guide professionals for better outcomes.

### SUMMARY OF THE INVENTION

In at least one embodiment, a system for orthopedic surgery is provided that includes a client device; a plurality of position sensor units configured to communicate position information wirelessly to the client device, where each of the position sensor units include an anchoring means for attaching position sensor units to bone; and an adjustable cutting block, where the cutting block an attachment portion having a recess therein for attaching the cutting block to a first of the plurality of sensor units; a cutting block portion having a second recess for attaching the cutting block to a second of the plurality of sensor units and an aperture extending through the cutting block portion for guiding a bone cutting instrument; and an intermediate portion coupling the attachment portion and the cutting block portion to each other, the cutting block therewith configured to adjustably set an orientation of the cutting instrument.

In at least one embodiment, the aperture is in a form of a slot with opposing planer surfaces for guiding an oscillating bone cutting saw blade.

In at least one embodiment, the cutting block portion further includes anchoring means for attaching the cutting block portion to bone and therewith fixing an orientation of the aperture therein when the cutting block portion is adjusted to a desired orientation.

In at least one embodiment, the aperture is in a form of a slot with opposing planer surfaces for guiding an oscillating bone cutting saw blade with respect to a cutting plane, and where fixing the orientation of the aperture sets the cutting block portion with respect to a desired cutting plane.

In at least one embodiment, each of the plurality of sensor units each have a common pre-defined shape that enables sensor unites to be interchangeable with respect to the cutting block first and second recess.

In at least one embodiment, the intermediate portion includes at least one pivot between the attachment portion and the cutting block portion.

In at least one embodiment, the intermediate portion includes at least one hinge between the attachment portion and the cutting block portion.

In at least one embodiment, the intermediate portion telescopically couples the attachment portion and the cutting block portion.

In at least one embodiment, the intermediate portion includes at least one pivot between the attachment portion and the cutting block portion.

In at least one embodiment, the intermediate portion further includes a hinge between the attachment portion and the cutting block portion and where the attachment portion and the cutting block portion are therewith adjustably coupled in three dimensions.

In at least one embodiment, the intermediate portion includes a rack and pinion configured between the attachment portion and the cutting block portion for setting a vertical distance between the attachment portion and the cutting block portion.

In at least one embodiment, the intermediate portion includes a hinge and a pivot between the attachment portion and the cutting block portion, the cutting block portion therewith adjustable vertically, rotationally about a vertical axis, and pivotally.

In at least one embodiment, at least one of the plurality of sensor units is a gyro sensor unit that communicates position information wirelessly to the client device.

In at least one embodiment, at least one of the plurality of sensor units is a magnetic sensor unit.

In at least one embodiment, the client device is configured to detect position information of each of the plurality of the sensors and construct a virtual model of a subject's skeletal anatomy based on kinematic data derived from position information from the plurality of sensors attached thereto.

In at least one embodiment, the client device is further configured to calculate at least one of a tibial and a femoral cutting plane based on individual kinematic data and muscular activity.

In at least one embodiment, the client device is further configured to display an interface screen depicting an avatar of the subject skeletal anatomy and superimpose the calculated cutting plane thereon.

In at least one embodiment, the client device is further configured to superimpose on the avatar an orientation of a guide cutting plane presented by the cutting block portion.

In at least one embodiment, the client device is further configured to calculate a thickness of the tibial cut.

In at least one embodiment, the client device is further configured to display a series of interface screens that queue users with respect to a resection workflow, including queues for placing position sensor units at predefined locations on a subject's ankle, hip, tibia, and femur.

In at least one embodiment, the queues further prompt users to attach the cutting block to at least one of the sensor units and adjust the cutting block portion relative to the attachment portion to achieve congruency with respect to a calculated cutting plane and a guide cutting plane presented by the cutting block portion.

In at least one embodiment, each of the calculated and guide cutting planes are presented as different colors until congruency is achieved.

In at least one embodiment, the client device further displays the avatar in a three-dimensional coordinate system and projects the cutting planes on at least one of the axial planes.

### BRIEF DESCRIPTION OF THE DRAWINGS

The invention is illustrated in the figures of the accompanying drawings which are meant to be exemplary and not limiting, in which like references are intended to refer to like or corresponding parts.

FIG. 1 illustrates a computing system according to at least one embodiment of the present invention.

FIG. 2-4 illustrate a three-dimensional adjustable cutting block according to at least one embodiment of the present invention.

FIG. 5 illustrates a hardware configuration of a three-dimensional orientation system according to at least one embodiment of the present invention.

FIGS. 6-10 illustrate sensor units configured at various anatomical locations according to at least one embodiment of the present invention.

FIGS. 11-35 illustrate exemplary software interfaces for measured resection techniques using the three-dimensional orientation system according to at least one embodiment of the present invention.

FIGS. 36-50 illustrate exemplary software interfaces for soft tissue balancing techniques using the three-dimensional orientation system according to at least one embodiment of the present invention.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the present invention. Likewise, a reasonably broad scope for claimed or covered subject matter is intended. Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of exemplary embodiments in whole or in part. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

The present application discloses a preferably imageless three-dimensional orientation system that determines and guides users with respect to correct implant positioning using individual patient kinematics data. The orientation system may include a plurality of wireless communicating single- or multi-use sensor units that when configured as disclosed herein may be able to replace conventional instruments. Arthroplasty surgery may be performed with the sensor units in conjunction with a three-dimensional adjustable cutting block, as also disclosed herein, and trial components that are specially designed to operate with the orientation system. As such, a surgeon may have the freedom to decide the alignment concept he will follow (e.g., anatomic, kinematic, mechanical), chose the type of procedure or technique (e.g., measured resection (“MR”), soft tissue balancing (“STB”), etc.), and have the option during surgery to switch to any conventional technique and setup in case of any doubt. As such, the presently disclosed system(s) enable performing arthroplasty on, e.g., the knee, that account for individual kinematics, kinetics, abilities and/or muscle status. Although the systems may be described herein in relation to arthroplasty with respect to the human knee, it is understood that they systems are equally useful for other joints and non-human subjects, and are therefore not limited thereto.

The disclosed systems preferably include a plurality of positioning sensors, e.g., six, which can be placed on pre-defined anatomical landmarks in a body area to be operated on, such as a knee joint, and one or more plane-marker sensors for fixing an adjustable cutting block relative to the joint bone being cut, preferably adjustable in three dimensions, in the desired position. The sensors may be optical, preferably non-optical, or a combination thereof. Optical sensors may include markers that are observed by system cameras, for example, to determine the position of the markers in a three dimensional space. Non-optical systems may include inertial units (referred to herein generally as “gyro” units), which may include at least one of a gyroscope, magnetometer, an accelerometer, or a combination thereof, to measure the position and/or orientation of each of the sensor units in three dimensions. Similarly, magnetic systems may be used to calculate position and/or orientation of the sensor unit based on the relative magnetic flux of the sensor units to three orthogonal coils. Together with an interface and a software application, the system may calculate the orientation of cutting planes on a tibial platform, enabling a proper physiological configuration based on individual patient kinematic data. The relation between the individual sensors together with their position in a three-dimensional space results in, e.g., a three-dimensional, trajectory system. Once defined, a user (e.g., surgeon) may choose a desired alignment concept (mechanical, anatomical or kinematic) as well as needed corrections in the case of post-traumatic or rheumatoid arthritis (“RA”) deformities.

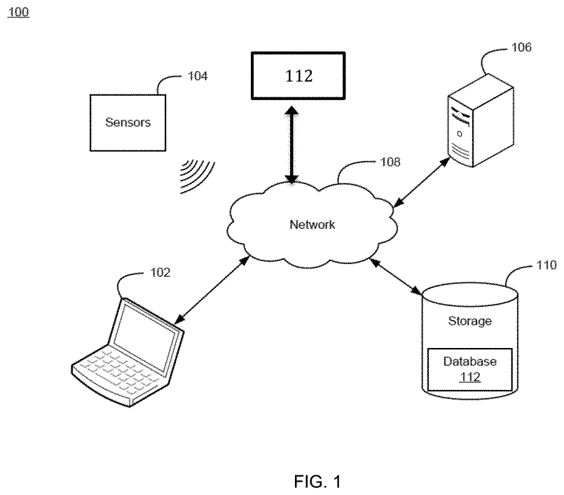

FIG. 1 illustrates a computing system according to an embodiment of the present invention. The system presented in FIG. 1, in accordance with at least one embodiment of the systems disclosed herein, includes one or more client device 102, a plurality of sensors 104, one or more servers 106, an at least partially wireless network 108, and/or remote storage 108. Depending on the type of sensor, the system may further include a receiver 112, such as a camera or magnetometer, which communicates position information obtained from or with the sensors 104 to the other systems components, over the network 108 or otherwise, as discussed herein. The receiver 112, e.g., a camera, may further detect gestures by users for hands free input by the surgeon, as explained below.

Client device 102 may include computing devices (e.g., desktop computers, terminals, laptops, personal digital assistants (PDA), cellular phones, smartphones, tablet computers, or any computing device having a central processing unit and memory unit capable of connecting to a network). The client device 102 may also include a graphical user interface (GUI) or a browser application provided on a display (e.g., monitor screen, LCD or LED display, projector, etc.). Client device 102 may also include or execute an application to communicate content, such as, for example, textual content, multimedia content, or the like, including content provided with the interface screens disclosed herein. The client device 102 may also include or execute an application or app to perform a variety of possible tasks, such as browsing, searching, playing various forms of content, including locally stored or streamed video, data input and output, etc. Client device 102 may include or execute a variety of operating systems, including a personal computer operating system, such as a Windows, Mac OS or Linux, or a mobile operating system, such as iOS, Android, or Windows Phone, or the like. Client device 102 may also include or may execute a variety of possible applications, such as a client software application enabling communication with other devices or components, such as communicating one or more messages, such as via email, short message service (SMS), or multimedia message service (MMS), including via a network.

Sensors 104 according to at least one embodiment include gyro sensor units that each include movement tracking, power, circuitry, and wireless communication components. The gyro sensors 104 may detect rotational motion and changes in orientation, and communicate that information to the system 100 for tracking the 3D position of the sensors 104 in three dimensions. The sensors 104 may be for single-use only and each have a defined size and shape to fit certain dimensions (e.g., 20×20×8 mm). The sensors 104 preferably include pins or other anchoring devices for attaching the individual sensor to the bones of the body part being operated on, such as a knee, and individually activated to send active signals to client device 102 either over network 108 or directly via short-range wireless communication (e.g., Bluetooth, Near-Field Communication, etc.). Each of sensors 104 may be activated by a magnetic switch, and identified or labeled according to their position relative to a plurality of predefined positions, and registered with an interface on client device 102.

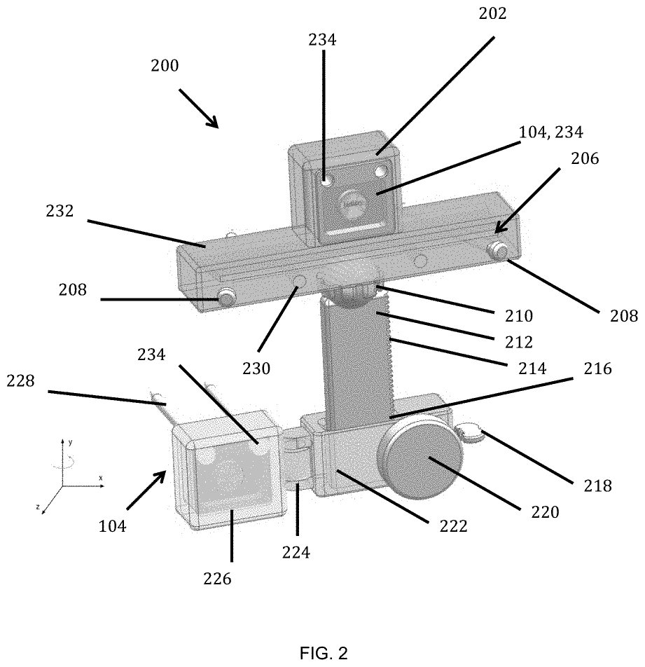

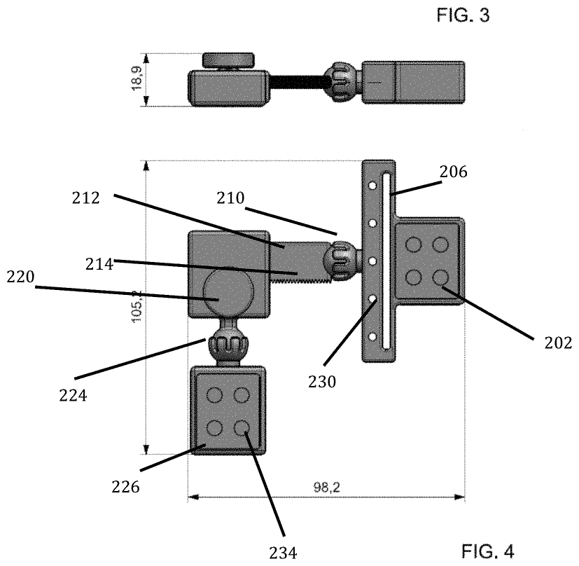

The sensors 104 in communication with client device 102 may, once activated, form a three-dimensional matrix or grid reference system such that an orientation of, for example, the tibial plane and the femoral joint surface can be modeled and validated with the system 100, as discussed further below. The system further includes a three-dimensional adjustable cutting block 200 (FIGS. 2-4), which is configured to attach to one or more of the sensors 104 previously affixed to bone, as shown in FIG. 3-4. The cutting block 200 includes one or more sensors 234 for determining the position and orientation of the cutting plane (e.g., plane-markers), which may be adjusted at specific relative locations to other ones of the sensors 104 to set up the desired individual orientation for cutting the tibial plane.

Referring to FIG. 2, the three-dimensional adjustable cutting block 200 may include cutting block portion 232, which includes a recess 202 therein for attaching thereto a plane-marker sensor 234 and an aperture 206 that sets the orientation of the plane for guiding a cutting instrument with respect to cutting bone in preparation for the implant. The aperture 206 may be a slot with opposing planer surfaces spaced apart from each other to guide an oscillating bone cutting saw blade, for example. The cutting block 200 may further include holes 230 for fixing the cutting portion 232 to the bone with, for example, pins 208 through holes 230, when the cutting block 232 is adjusted to achieve the desired cutting plane orientation. The cutting block 200 further includes a first attachment portion 226 that has a recess therein for attaching the block 200 to a sensor 104, which has been affixed previously to the bones of a joint, e.g., with pins 228 passing through holes 234 in the sensor 104, as discussed herein. As discussed above, the sensors have a defined shape and size so that the cutting block 200 can be attached to any one of the sensors 104, and thus the sensors 104 can be used interchangeably prior to registration with the system.

The first attachment portion 226 and cutting block portion 232 are adjustable relative to each other, preferably in three dimensions. This may be achieved in a variety of ways. As shown in FIG. 2, articulation in this regard may be achieved with an intermediate portion that includes one or more swivel connections 210, 224, hinges 224, and/or a telescopic mechanism(s), such as rack 212 and pinion 222. The rack 212 and pinion 222 system may include an adjuster 220 with teeth 216 thereon that engage corresponding teeth 214 on rack 212 to adjustably extend or retract cutting block portion 220 telescopically, i.e., in the vertical (y) direction. This “height” may be locked into place with a locking screw 218. The adjustability of the cutting plane may be enabled with hinge 224 or/or with swivels 210, 224. In the embodiment shown in FIG. 2, the cutting plane presented by block 232 is adjustable vertically, rotationally about the vertical axis, and pivotally.

The three-dimensional adjustable cutting block 200 may have an exemplary length and width of about 98.2 mm×105.2 mm, but may be any dimension suitable for the joint at issue. A portion of the three-dimensional adjustable cutting block including the adjuster may have an exemplary thickness of about 18.9 mm. In at least one embodiment, as shown in FIGS. 3-4, the adjustable cutting block includes a first attachment portion 226 coupled to an intermediate telescopic portion, which is further coupled to the cutting block portion 232. The first attachment portion 226 and the cutting block portion 232 preferably each include the same sized compartment for receiving and attaching to at least one sensor therein. The intermediate portion is preferably coupled to the other portions with a hinge or pivot that allows the user to adjust the cutting portion relative to the first attachment portion. The intermediate portion preferably includes a pair of structure operable to orthogonally slide relative to each other, which allows the user to adjust the distance between the sensors, and correspondingly the distance of the cutting plane relative to the sensor to which the cutting block 200 is attached.

Server 106 may include a computing system operable to allow access to storage 110 over network 108. Network 108 may be any suitable type of network allowing transport of data communications across thereof. The network 108 may couple devices so that communications may be exchanged, such as between server 106 and client device 102 or other types of devices, including between wireless devices coupled via a wireless network, for example. The network 108 may also include mass storage, such as network attached storage (NAS), a storage area network (SAN), cloud computing and storage, or other forms of computer or machine-readable media, for example. In one embodiment, the network 108 may be the Internet, following known Internet protocols for data communication, or any other communication network, e.g., any local area network (LAN) or wide area network (WAN) connection, cellular network, wire-line type connections, wireless type connections, or any combination thereof.

Server 106 may include at least a special-purpose digital computing device including at least one or more central processing units and memory. The server 106 may also include one or more of mass storage devices, power supplies, wired or wireless network interfaces, input/output interfaces, and operating systems, such as Windows Server, Mac OS X, Unix, Linux, FreeBSD, or the like. In an example embodiment, server 106 may include or have access to memory or computer readable storage devices for storing instructions or applications for the performance of various functions and a corresponding processor for executing stored instructions or applications. For example, the memory may store an instance of the server configured to operate in accordance with the disclosed embodiments.

Server 106 is operative to receive requests from client device 102 to retrieve analysis and procedure data of a patient including a virtual model of a given body part from database 112. Prior to surgery, a preliminary examination may be performed with optical markers and electrodes attached to wearable stockings (or any other wearable article) to identify a target patient's movement and kinematic data. This may be done with a special movement analysis with 3D cameras together with an electromyography (“EMG”) measurement to value the activity of the individual muscles of the joint. While wearing the stocking, the patient may be analyzed during walking on a treadmill, performing stairs up and down, and moving the knee against a defined resistance in flexion and extension. Accordingly, different values of muscular activity may be recorded and analyzed by either client 102 or server 106. These values can be compared to the other side (contralateral), ideally the healthy one. If the contralateral side is also affected, a template database of healthy patients (stored in database 112) may be used for comparison.

The analysis may be performed, together with long leg x-ray from the front (coronal plane) and the side (sagittal plane), to produce a three-dimensional active avatar of a whole leg or body part. The avatar may include a virtual leg moving with respect to individual muscle activity and the anatomical situation of the joint capsule, ligaments and bones. Such a virtual model can be used to virtually implement the prosthesis and determine the correct three-dimensional orientation of the tibial plane. The data generated for the avatar may be the basis for a correct cutting plane with respect to a tibial baseplate and may be stored to database 112. The virtual model when used in conjunction with an anamnesis of the progress of disease, a surgeon can decide which protocol to follow.

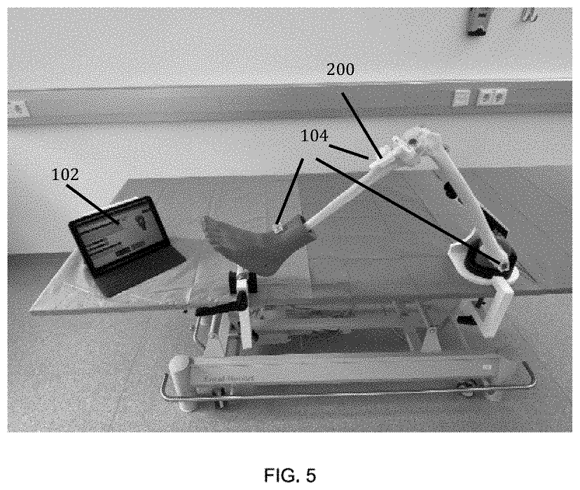

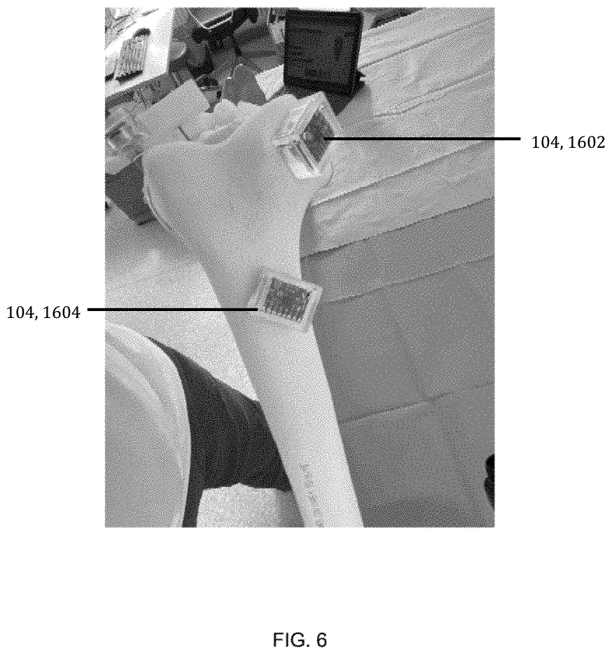

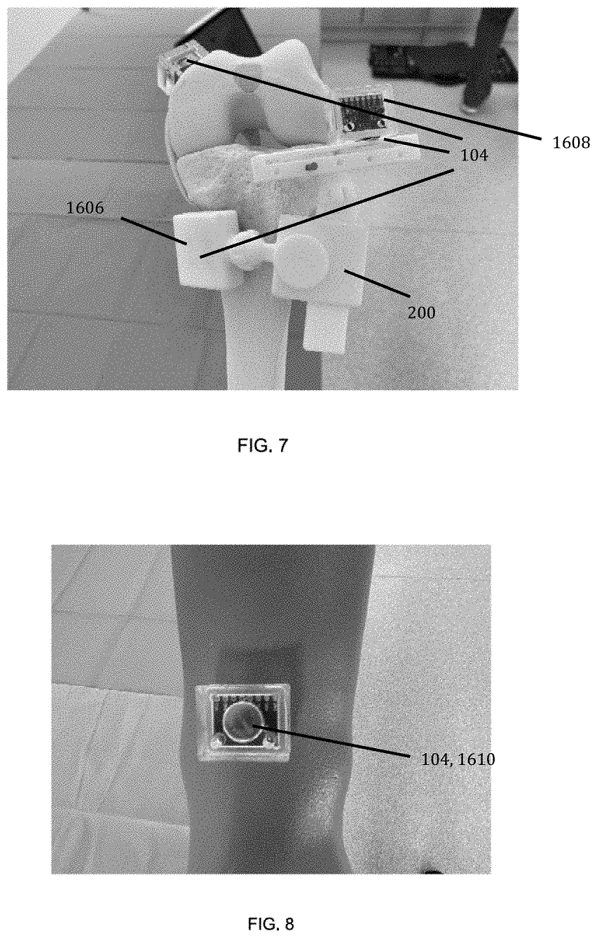

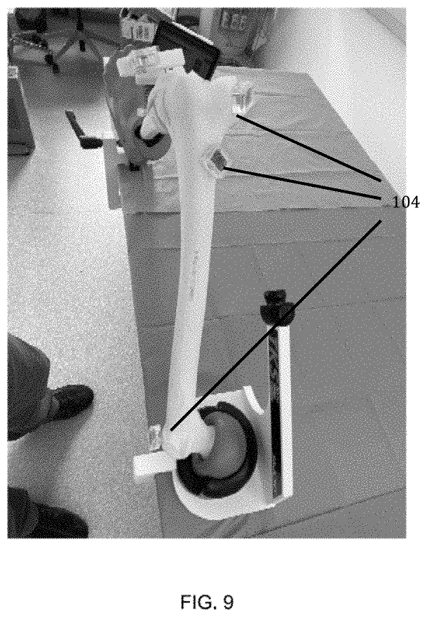

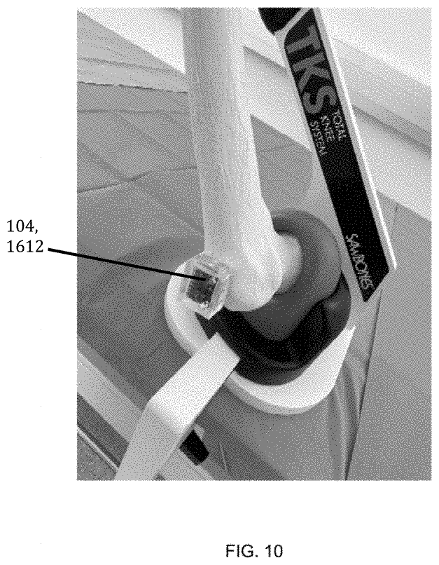

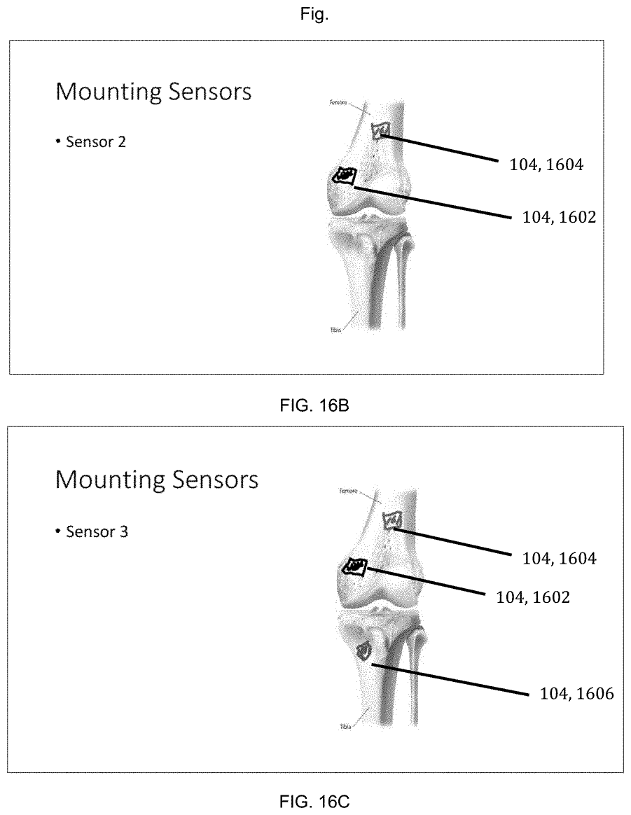

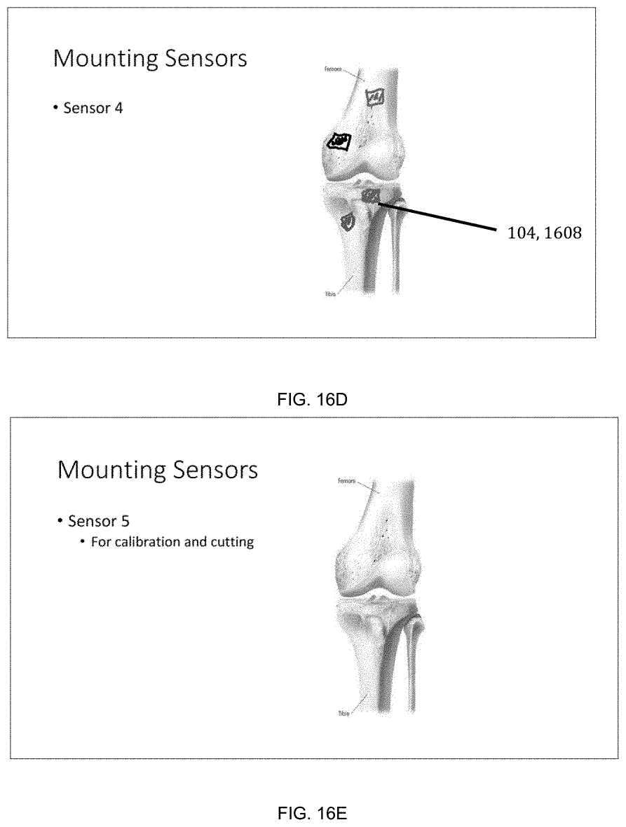

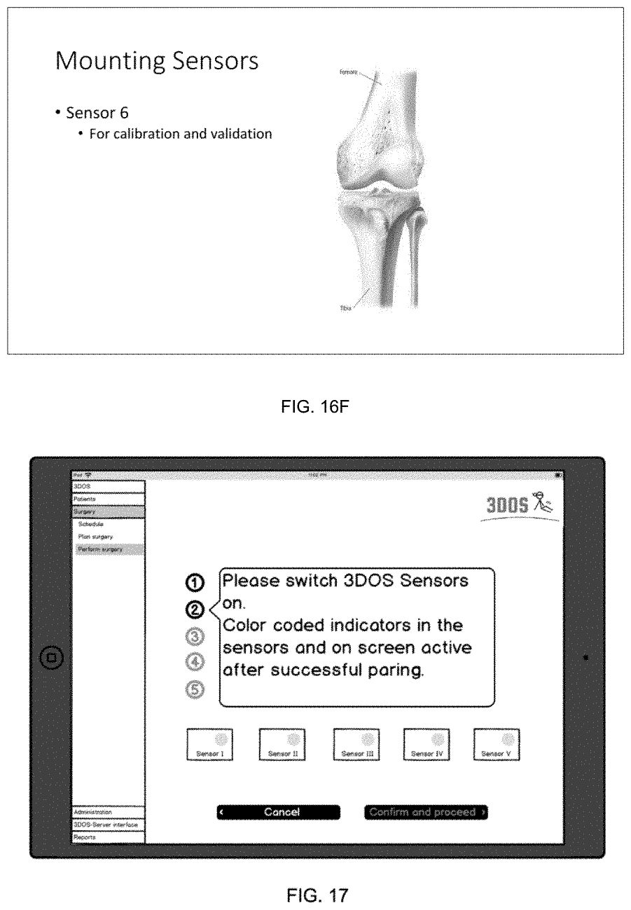

FIGS. 5-10 present a hardware configuration of a three-dimensional orientation system according to an embodiment of the present invention located on a partial skeleton as an illustration of the system in use. A client device 102 may be configured to communicate with sensors 104 installed in certain positions on the bone of body part being operated on. In one example, after opening to expose the knee joint, ideally preserving all functional structures, especially the muscle envelope, sensors 104 may be placed on defined landmarks on the subject's femur (FIG. 6), tibia (FIG. 7), anterior aspect of the ankle (FIG. 8), and at the trochanteric region (FIGS. 9 and 10). On the femur, sensors 104 may be placed on the base of the trochlea groove near the cartilage bone border (a first sensor 1602), and on the area between the medial epicondyle and the cartilage-bone border with orientation to the joint (a second sensor 1604), as shown in FIG. 6 and FIGS. 16A-16B. On the tibia, a third sensor 104, 1606 may be placed on the medial border of the tibial tuberosity, as shown in FIG. 7 and FIG. 16C. When cutting the tibia, the cutting block 200 may be attached to the third sensor 104, 1606 and a fourth sensor 1608 attached to the cutting block 200, as shown in FIG. 7 and FIG. 16D. These sensors 104 are preferably secured to the femur or tibia so that they do not interfere with the cutting planes established with the cutting block 200. Finally, fifth 1610 and sixth 1612 sensors may be attached to the tibia near the ankle and on the femur near the greater trochanter, respectively, as shown in FIGS. 8-10.

The placements of the sensors 104 on the subject's skeletal anatomy generally enables the system to create a model of the skeletal anatomy in a three-dimensional reference grid or system, i.e., a 3D model, which may be generated by the client device 102 and/or server based on the location information obtained by the client device 102 and/or server from respective sensors 104 communicating with the client device 102 and/or server. In case of preoperative planning, the three-dimensional model may be compared with a preoperatively calculated reference model. The model generated by the system preferably includes location information of each of the sensors 104 throughout the range of movement of the joint. In this regard, location data capture may include performing several flexion and extension movements of the joint until a software interface of the client device 102 shows that the system acquired the data necessary to generate the 3D model of the joint, e.g., three-dimensional positioning of the knee joint, through at least a portion of the range of movement of the joint.

An orientation screen may be presented or otherwise displayed by the client device 102 to guide the use in setting up the proper orientation of the three-dimensional adjustable cutting block 200 and more specifically the cutting block portion 232 thereof. Beside the orientation of the plane, the thickness of the cut may be determined by the client device 102 such that the bone loss can be minimized as much as possible. When the orientation and/or thickness of the cut is verified by the system based on information from the sensor attached to the cutting block portion 232, the three-dimensional cutting block 200 may be fixed by pins for maximum available stability during the cutting. When the surgeon has verified the plane and/or thickness of the cut is proper, the cut of the tibial plane can finally be performed. After cutting the tibial plane, the resulting plane can be compared to an initial planning using a dynamic ligament balancing device, as discussed in commonly-owned U.S. Patent Publication No. 20170312099 which is incorporated herein by reference, and may include another sensor that increases the accuracy of the measurement with respect to the desired final outcome. In either event, the arthroplasty surgery can be completed normally.

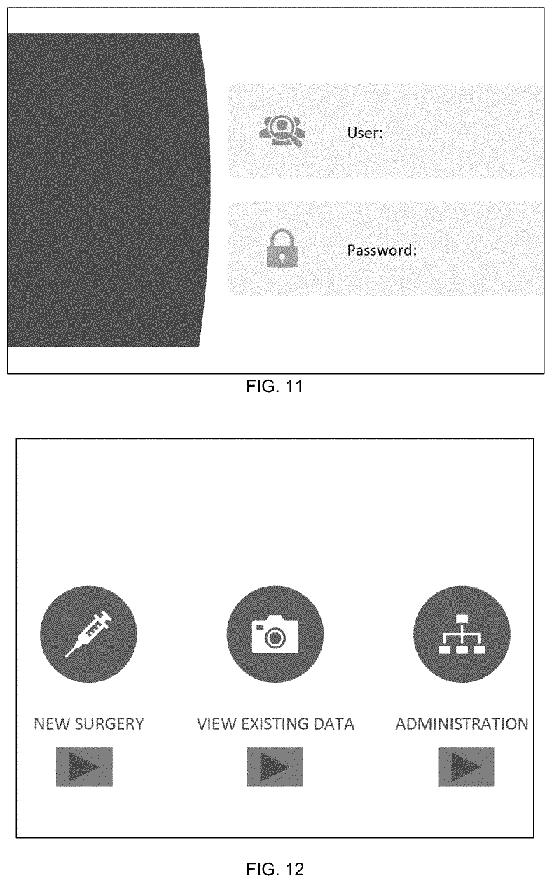

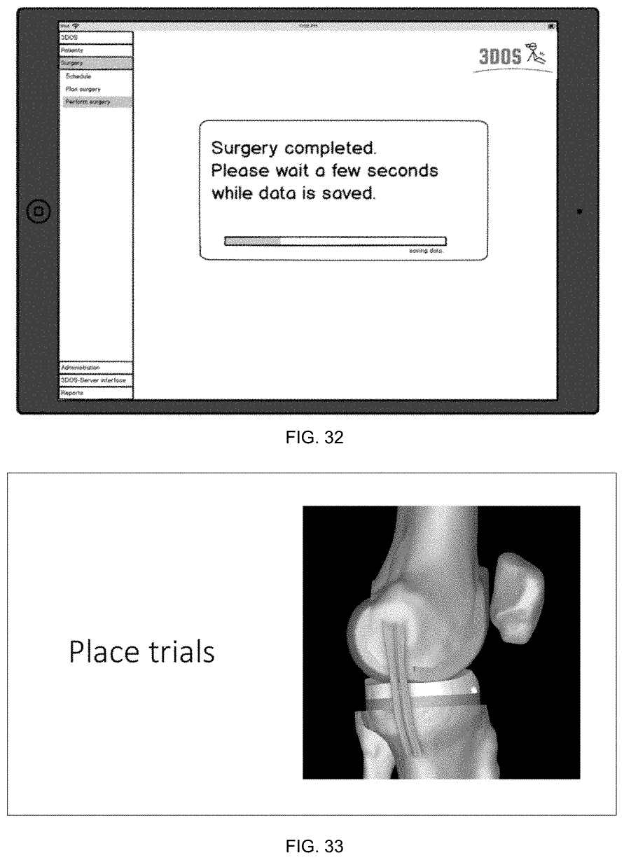

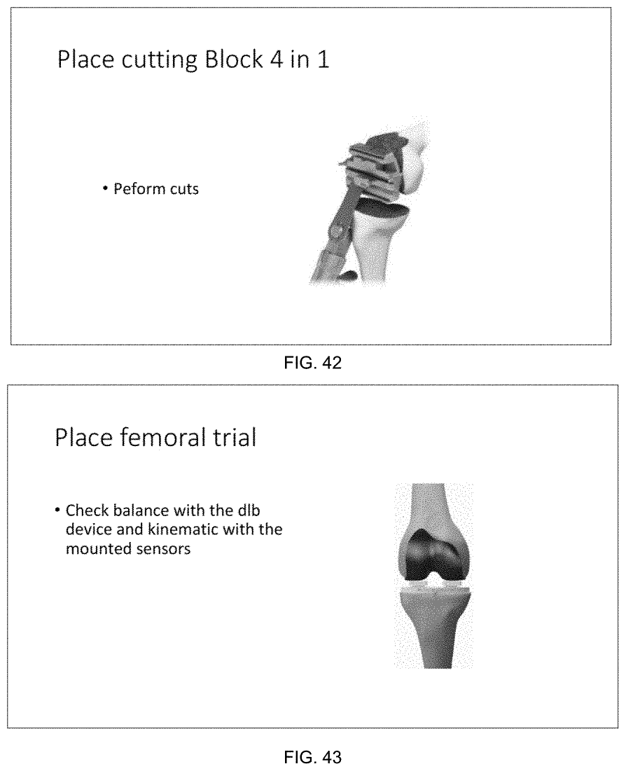

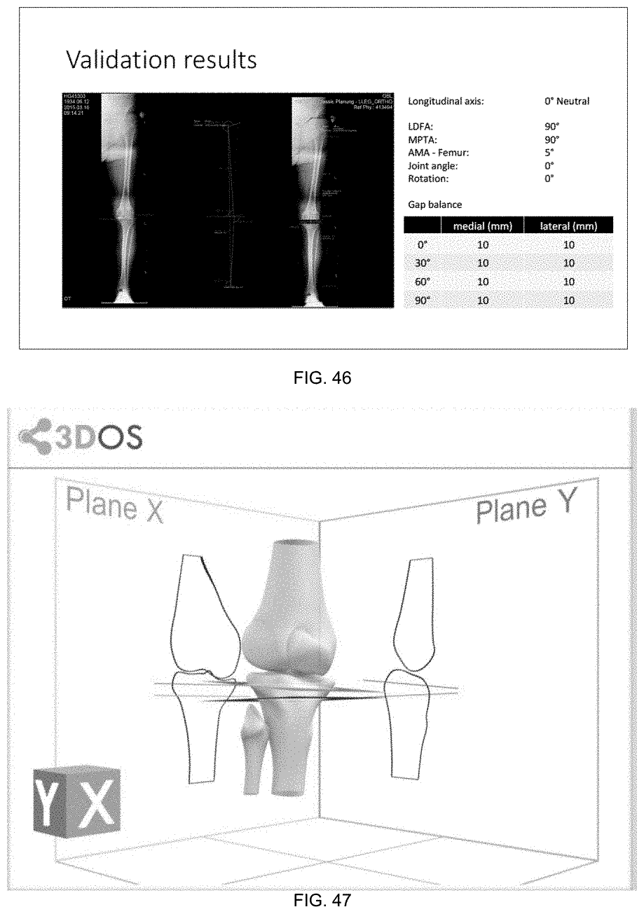

FIGS. 11 through 50 present exemplary software interfaces for use in conjunction with the three-dimensional orientation system according to the embodiments discussed herein. The interfaces may be loaded or otherwise displayed on a client device for data acquisition and presentation as discussed herein. In one embodiment, the system provides a user with options for logging on, such as with a user name or email address, and password (FIG. 11). A welcome screen may be provided to display information, such as statistics, the user's surgery schedule, information updates, etc. An interface may be presented (FIG. 12), which provides users options for entering information for new surgeries, or enter patient data to retrieve existing preoperative and postoperative information, including data, examinations, x-rays, and one or more virtual models of the skeletal anatomy from the relevant database(s). The user may also be allowed to view information based on a selection of a type of prosthesis, location, alignment, size, and also the type of procedure or technique (MR/STB).

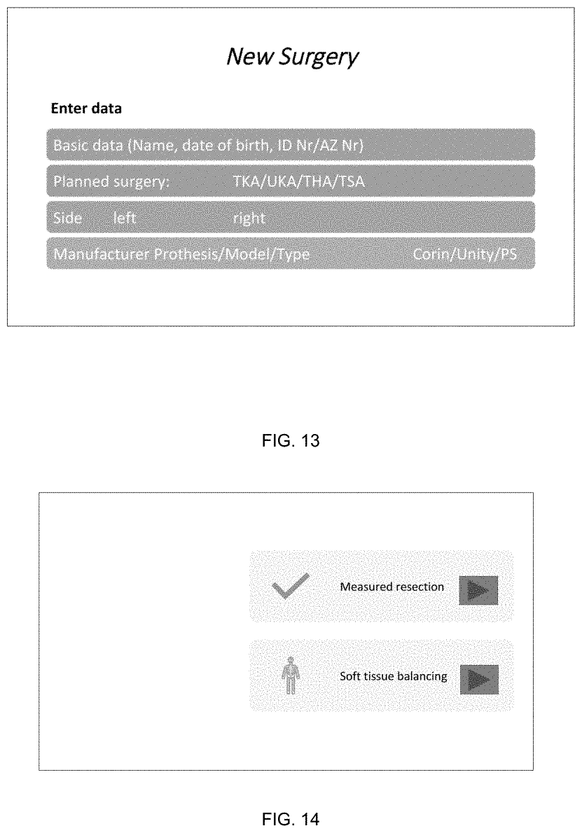

If the user selects a new surgery, the system preferably displays an interface with form elements therein, such as text boxes and/or selectable lists, for the user to enter the data for the surgery, such as basic patient information (name, date of birth, ID number, etc.), the surgery being planned (TKA (total knee replacement)/UKA (unicompartmental)/THA (total hip arthroplasty)/TSA (total shoulder arthroplasty)), the side of the surgery (left/right), and/or the prosthesis information (manufacturer, model, type), as shown in FIG. 13. Once the patient/surgery information is uploaded and/or updated, the system may present an option to perform a measured resection and/or soft tissue balancing, as shown in FIG. 14.

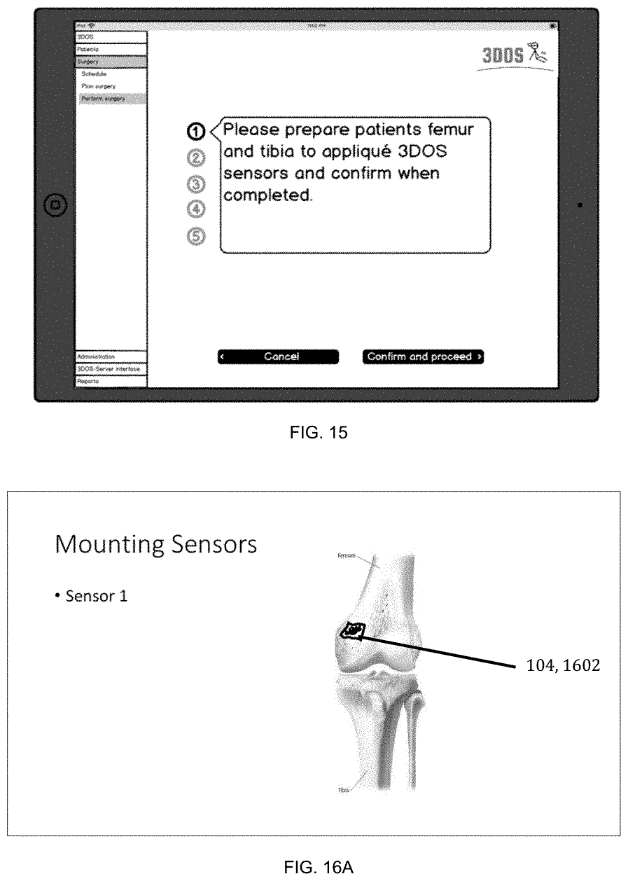

Selecting measured resection in FIG. 14 may result in a first of a series of interface screens that provide users with queues and allow users to input information for the resection workflow. As shown in FIG. 15, the first interface screen may prompt the user to prepare the patient for the application of the sensors 104. This interface screen may include buttons for the user to confirm that the first general step of the workflow has been performed.

In at least one embodiment, the system may guide the user with respect to attaching the sensors (for example, 3-6, or more) to the subject's skeletal anatomy and registering each of the individual sensors 104 with the system. For example, as shown in FIG. 16A, the system may display a representation of the joint and the location of the first sensor. Once the first sensor 1602 is attached, the workflow may be repeated for each of the remaining sensors, as shown in FIGS. 16B-16F. When all of the sensors are attached, the system may prompt the user to activate the sensors and may thereafter pair the sensors with the system, as shown in FIG. 17. The interface screen may include a pair status for each of the sensors, e.g., green paired vs. red unpaired.

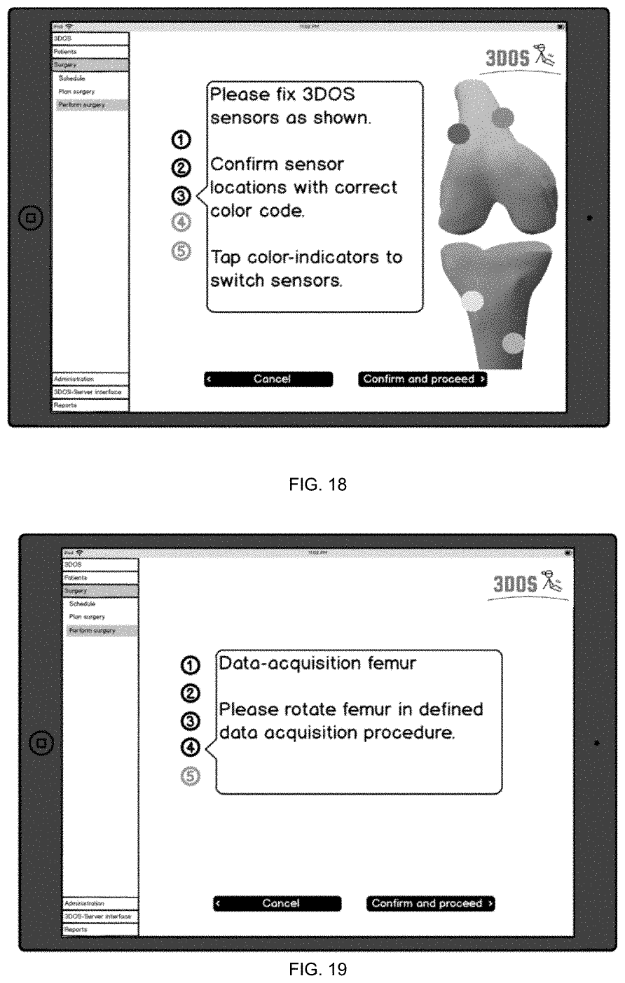

In one embodiment, once all of the sensors are paired, the system may prompt the user to associate or confirm the association of each of the sensors with the predefined locations. As shown in FIG. 18, each of the sensors may have an address represented by a color code. If the color code of the sensor does not match the color displayed on the interface screen, the system may provide options for the user to change the color of each of the sensors displayed on the interface screen. For example, the sensors displayed on the screen may be enabled as buttons that once selected drop down a list of color options for the user to specify the correct sensor color at the predefined location.

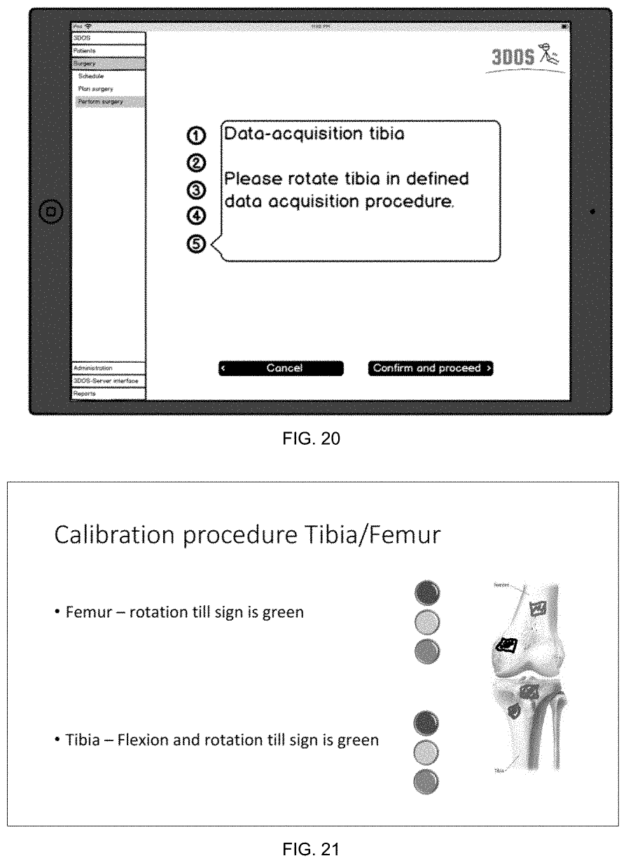

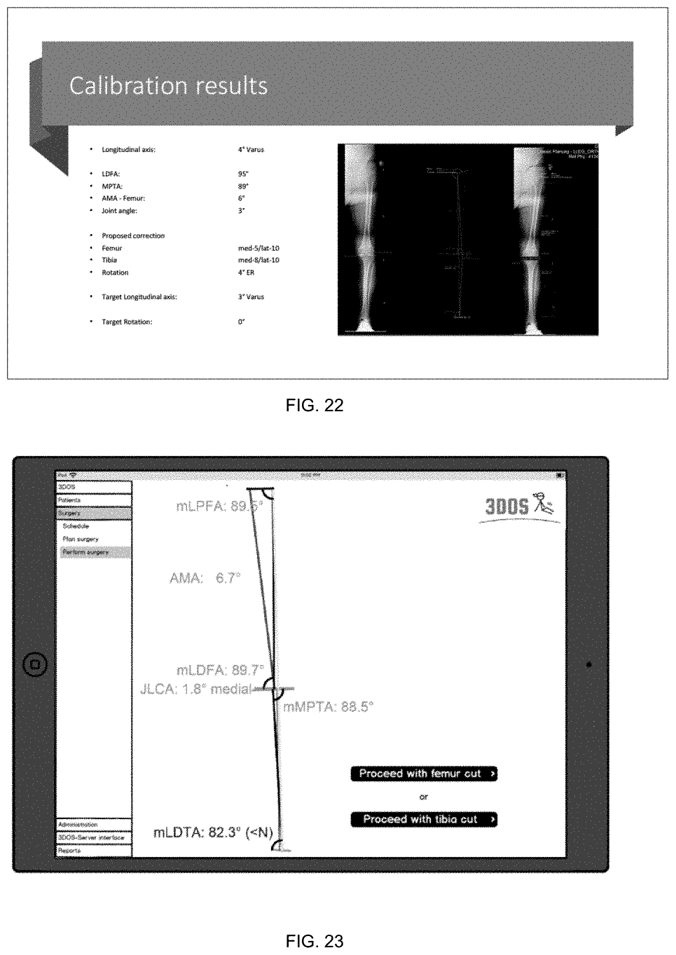

Once the location of the sensors is confirmed, the system may prompt the user to rotate the bones of the subject's joint for data acquisition, as shown in FIGS. 19-20 with respect to the femur and tibia. In a preferred embodiment, the interface screen may display indicia with respect to the status of data acquisition. For example, red/yellow/green may be displayed in sequence to indicate the status of data acquisition as the bones of the joint are moved through their range of motion, as shown in FIG. 21. The data acquired generally includes the 3D location of each of the sensors through the desired range of motion, based on, for example, the gyroscope, magnetometer, an accelerometer, or a combination thereof, of each of the sensors. The system stores the data in one or more databases and subsequently uses the sensor location dataset to generate a model of the skeletal anatomy of the subject in the three-dimensional reference grid or system, as discussed above. This preoperative model data is preferably used to calculate variables associated with joint/bone alignment, as shown in FIGS. 22-23, including the orientation of the cutting plane and/or thickness. Once calculated, the user may proceed to a cutting workflow by selecting the appropriate button in the interface screen, as shown in FIG. 23.

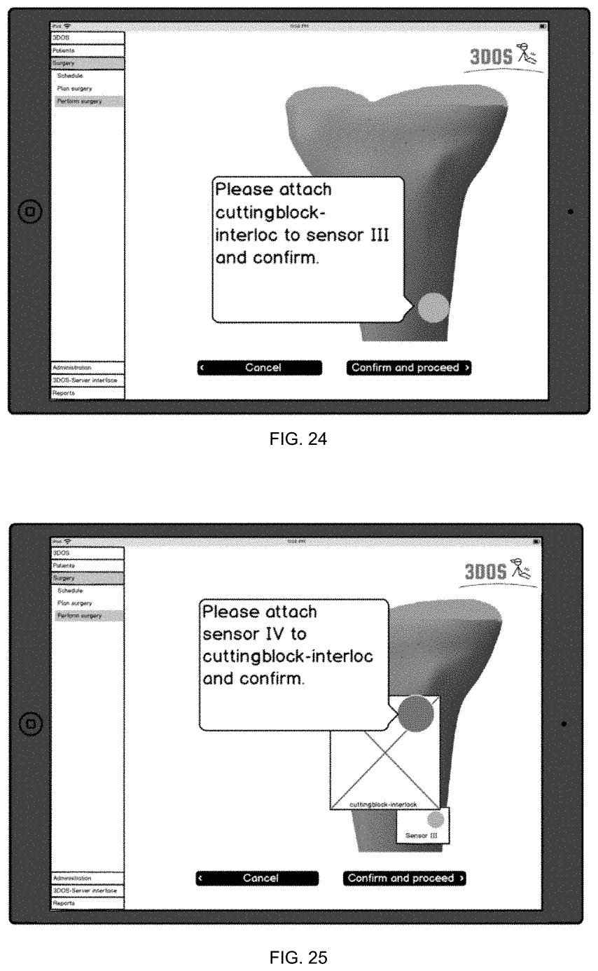

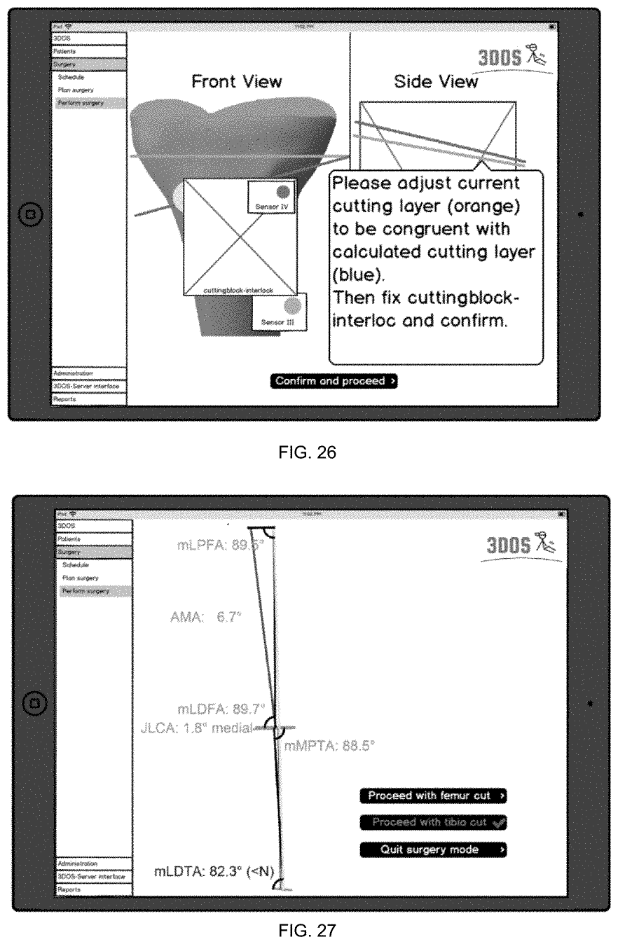

Selecting the option to proceed with the tibia cutting may cause a series of interface screens that guide the user with respect to aligning the cutting plane to achieve the desired post operative alignment. For example, as shown in FIG. 24, the system may prompt the user to attach the cutting block 200 to the appropriate sensor at the joint, such as sensor 3 (1606) on the tibia. If not already present in the cutting block, the system may prompt the user to attach sensor 4 (1608) to the cutting block 200 and more specifically the cutting block portion 232, as shown in FIG. 25. Once confirmed, the system may display an interface screen that depicts the orientation of the calculated cutting plane in a first color, for example blue, and preferably the orientation of the guide surfaces of the aperture in the cutting block portion 232 in a second color, for example orange, as shown in FIG. 26. The system preferably refreshes the interface screen in real-time as adjustments to the cutting block portion 232 are made to confirm when the actual cutting plane is congruent with the calculated cutting plane. Once congruency is achieved, the surgeon may fix the cutting block portion 232 to the bone for stability during cutting, and proceed with the first cutting, e.g., of the tibia, the results of which may be displayed in an interface screen, as shown in FIG. 27.

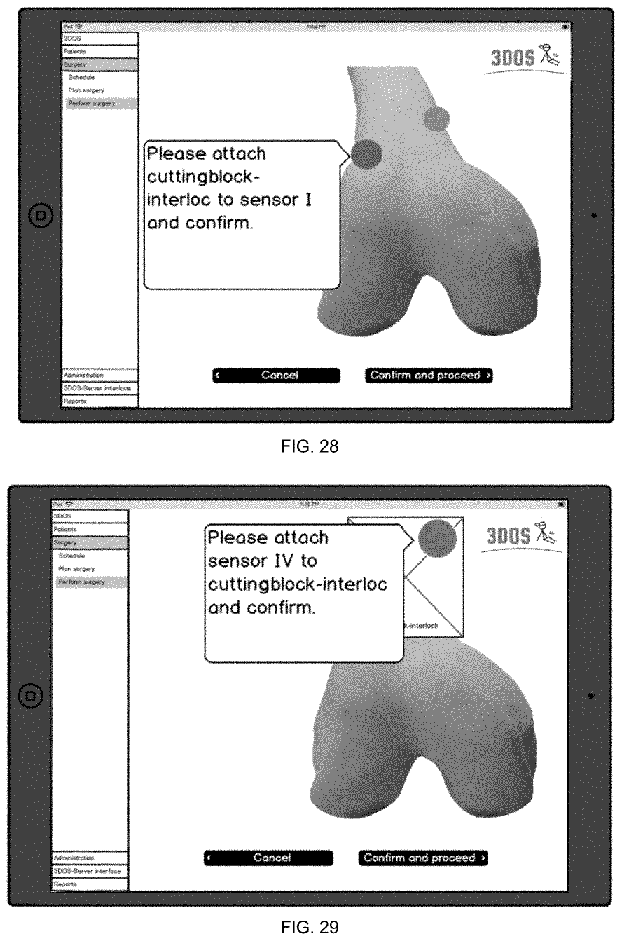

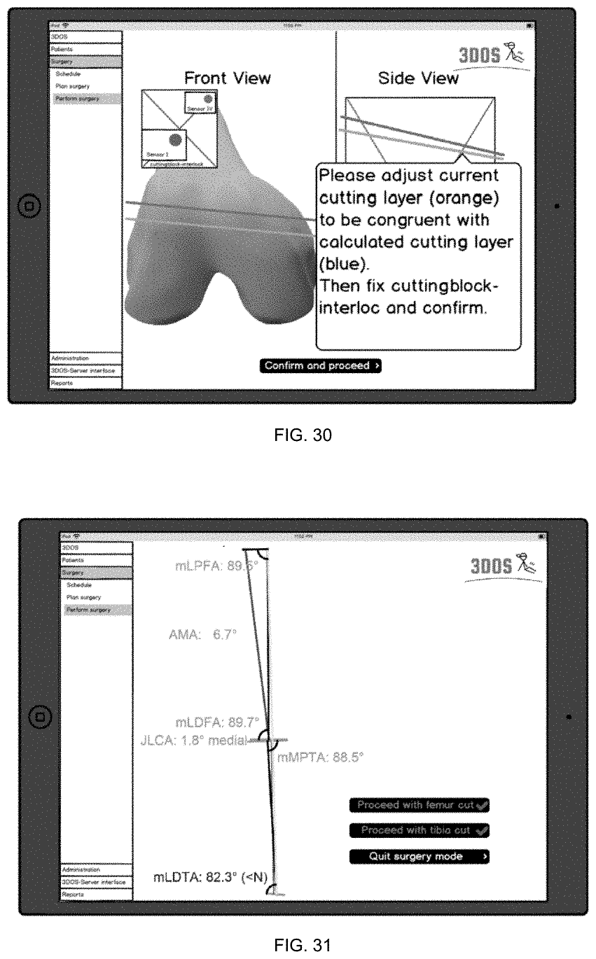

Selecting the option to proceed with the femur cut, as either the first cut as in the flow beginning in FIG. 23 or the second cut as in the flow beginning in FIG. 27, the system may in response display an interface screen that directs the user to install the cutting block to a sensor on the femur, such as sensor 1 (1602), as shown in FIG. 28. If not already present in the cutting block, the system may prompt the user to attach sensor 4 (1608) to the cutting block portion 232, as shown in FIG. 29. Thereafter, the system may present an interface screen that depicts the orientation of the calculated cutting plane in a first color, for example blue, and preferably the orientation of the guide surfaces of the aperture in the cutting block portion 232 in a second color, for example orange, as shown in FIG. 30, except this time for the femur. The system preferably refreshes the interface screen in real-time as adjustments to the cutting block portion 232 are made to confirm when the actual cutting plane is congruent with the calculated cutting plane.

The calculated and cutting planes may be displayed in the interface screens in three dimensions, superimposed over a 3D model of the joint at issue, as shown in FIGS. 47-50.

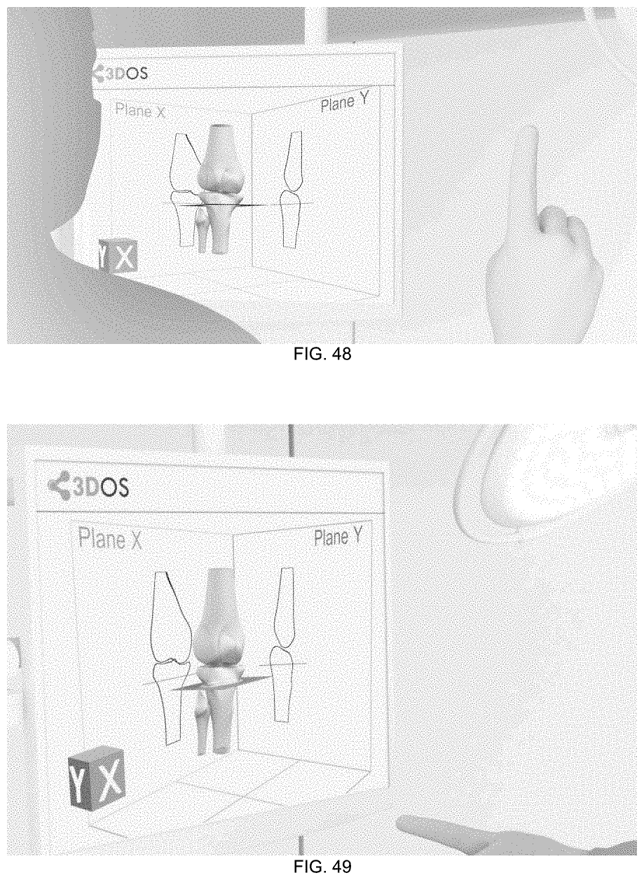

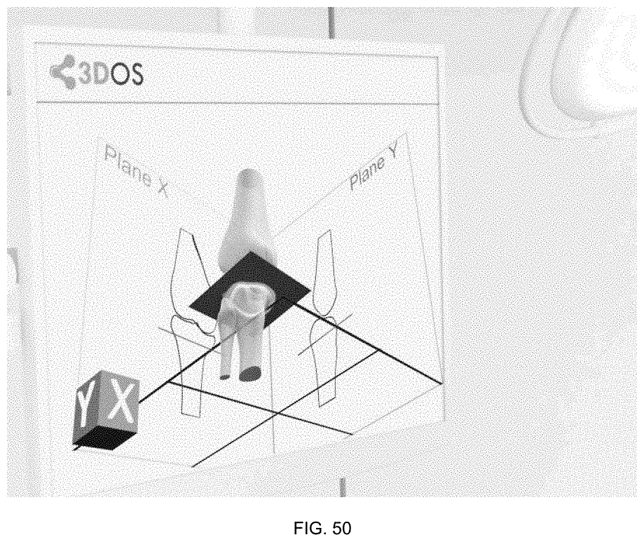

In this embodiment, the user is preferably able to view the model from any vantage point by rotating the reference system about any of one or more of the x-y-z axis, as shown. To aid adjusting the cutting block portion 232 for setting the cutting plane, the system may project the cutting planes and/or the calculated planes onto the coordinate planes, as shown in FIG. 47 showing projections on the X-plane and Y plane. As discussed above, the system may include hands-free functionality for user control of the orientation of the 3D model views. For example, the system may include a camera that observes the user rotating a vertical finger, which correspondingly causes the 3D model to rotate about the z-axis, as shown in FIG. 48. Similarly, observing the user rotating a horizontal finger may result in a corresponding rotation of the 3D model about an axis on a horizontal plane, as shown in FIGS. 49-50.

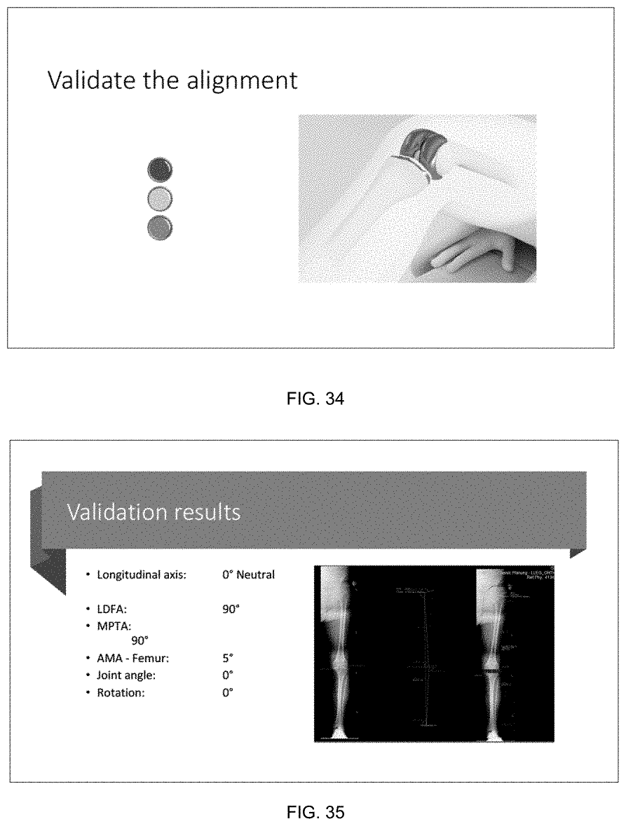

In one embodiment, the system may further the alignment of prosthesis and allow the user to adjust the implant as necessary to achieve the desired alignment. Specifically, the user may be presented with a “place trials” interface, as shown in in FIG. 33, which prompts the user to move the tibia and/or femur with the sensors thereon for the use to collect a trial prosthesis or post-implant dataset, and display the validation results, as in FIG. 35. The validation interface screen may further include indicia showing a successful data collection for validation, such as red/yellow/green light icons or graphics, as shown in FIG. 34.

Generally, interface screens show the user the position of where sensors should be placed on the bones, an order of sensor activation, and an indication of proper function with the sensors. A calibration process may be initiated by detected rotational movements of the femur and the tibia by sensors, as shown in the accompanying drawings. After finishing the calibration, a popup may show a progress of calculation and the achieved data may be shown on the interfaces noted above. In a preferred embodiment, the calculated cutting plane may be shown on an avatar of a body part via the interface on the client device to create a three-dimensional reconstruction or model including the parts of the prosthesis. The 3D reconstruction may include the trajectories of the anatomical landmarks during flexion-extension of the subject's bones. Functional stability, tension status and contractility of the muscles may be calculated to approximate the result as near as possible to a healthy situation. Special requirements and potential failure sources may also be identified using the systems disclosed herein.

The interface on the client device in communication with sensors may present a comparison of trajectories on the avatar with an intraoperative situation to check the overlap of the different trajectory curves to confirm synchronization among the avatar and the patient. The interface may generate coordinates with reference to the avatar and to the intraoperative placed sensors. This correlation may define the exact intraoperative position of the proximal tibial cutting plane. The user may then choose a first bone for the initial cut (femur or tibia). A picture of the bone may be shown displaying the correct orientation of the cutting plane. The three-dimensional adjustable cutting block can be mounted on a marked or indicated sensor (on the interface) and a control sensor may be fixed on the three-dimensional adjustable cutting block to adjust to an appropriate orientation plane, as shown and discussed above. When the orientations between the calculated and desired cutting planes are matching, the block can be fixed and the cut of the bone can be performed. Data from a completed surgery procedure may be saved to a database at a server or locally on the client device.

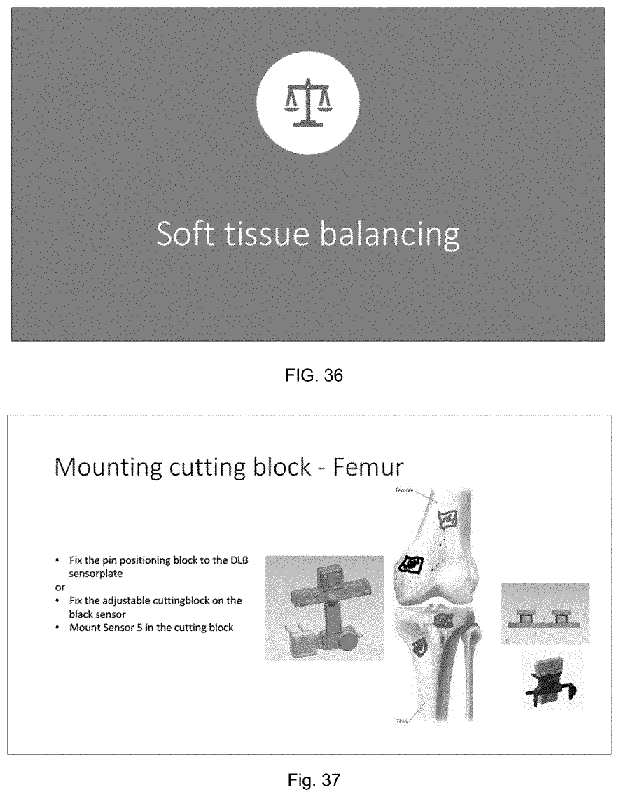

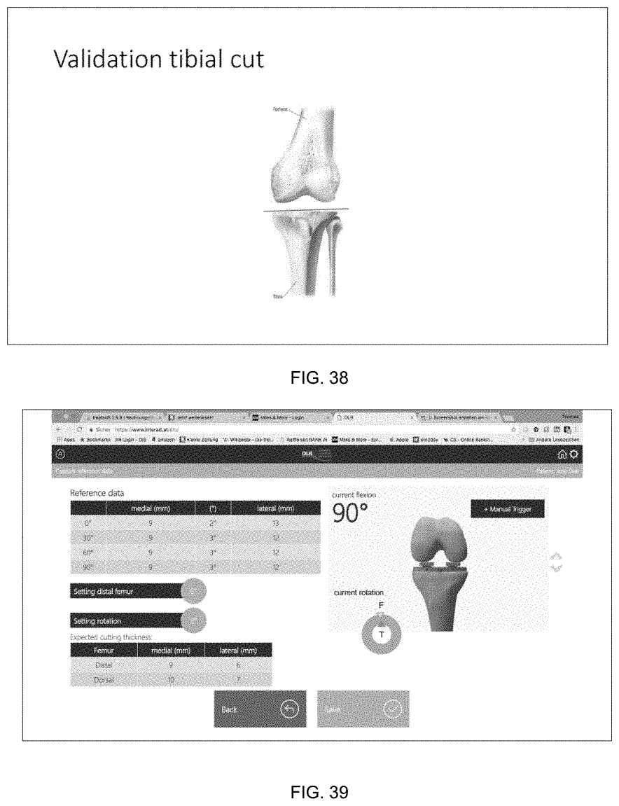

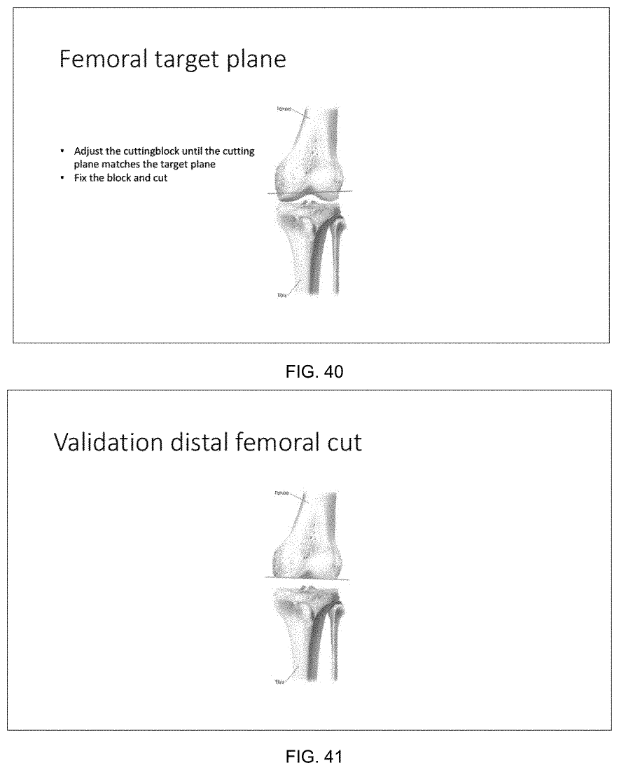

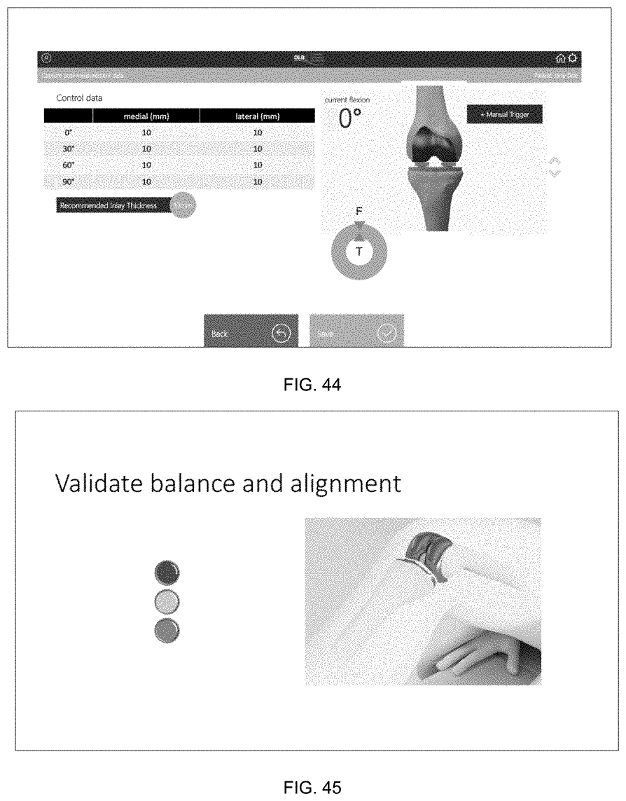

As discussed above, the systems disclosed herein may be used in combination with the dynamic ligament balancing sensor which is described in further detail in commonly owned U.S. Patent Application Publication No. 2017/0312099. In the case of the soft tissue balancing workflow (FIG. 36), the system may prompt the user to perform a tibial cut and validate the cut as discussed above and as shown in FIGS. 37-38. Once the tibia cut is performed, the dynamic balancing device may be placed between the cut and the femur, as shown in FIG. 39 and pre-prosthesis displacement data sets obtained at various angles of a current rotation. Thereafter, the femoral cuts may be performed (FIGS. 40-42) and femoral trials installed with the dynamic ligament balancing sensor between the trial and the tibial cut, as shown in FIG. 43. Finally, the trials displacement data set may be obtained and used to validate soft tissue balance and alignment, as shown in FIGS. 44-46.

Generally, with the measured resection procedure, the interfaces displayed by the system may present the correct orientation of the femoral cutting plane and the three-dimensional adjustable cutting block may be mounted again together with additional ones of sensors for orientation control. Again, after matching the shown plane with an adjusted cutting plane, the three-dimensional adjustable cutting block may be fixed and the cut can be performed.

In the case of a measure resection procedure, the axis orientation can be checked and the rotational adjustment of the three-dimensional adjustable cutting block on the femur can be performed. Pins can be inserted in the bony surface and the original three-dimensional adjustable cutting block of the used prosthesis can be mounted to perform the cuts. In case of STB, the sensor can be placed on the tibial surface and the pin positioning block of the three dimensional adjustable cutting block may be connected to the sensor plate to continue with setting the pins for the longitudinal and rotational alignment in place. Cuts may be performed, the trials may be put in place, and the control measurement can follow. The interfaces may show the orientation of the planes comparing to the initial measurements for quality control, as discussed herein.

FIGS. 1 through 50 are conceptual illustrations allowing for an explanation of the various embodiments of the present application. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

It should be understood that various aspects of the embodiments of the present invention could be implemented in hardware, firmware, software, or combinations thereof. In such embodiments, the various components and/or steps would be implemented in hardware, firmware, and/or software to perform the functions of the present invention. That is, the same piece of hardware, firmware, or module of software could perform one or more of the illustrated blocks (e.g., components or steps). In software implementations, computer software (e.g., programs or other instructions) and/or data is stored on a machine-readable medium as part of a computer program product and is loaded into a computer system or other device or machine via a removable storage drive, hard drive, or communications interface. Computer programs (also called computer control logic or computer-readable program code) are stored in a main and/or secondary memory, and executed by one or more processors (controllers, or the like) to cause the one or more processors to perform the functions of the invention as described herein. In this document, the terms “machine readable medium,” “computer-readable medium,” “computer program medium,” and “computer usable medium” are used to generally refer to media such as a random access memory (RAM); a read only memory (ROM); a removable storage unit (e.g., a magnetic or optical disc, flash memory device, or the like); a hard disk; or the like.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

What is claimed is:

1. A system for orthopedic surgery, comprising:
a client device;
a plurality of position sensor units configured to communicate position information wirelessly to the client device, wherein each of the position sensor units comprises an anchoring means for attaching position sensor units to bone; and
an adjustable cutting block comprising:
an attachment portion having a recess therein for attaching the cutting block to a first of the plurality of sensor units;
a cutting block portion having a second recess for attaching the cutting block to a second of the plurality of sensor units and an aperture extending through the cutting block portion for guiding a bone cutting instrument; and
an intermediate portion coupling the attachment portion and the cutting block portion to each other, the cutting block therewith configured to adjustably set an orientation of the cutting instrument.

2. The system of claim 1, wherein the aperture is in a form of a slot with opposing planer surfaces for guiding an oscillating bone cutting saw blade.

3. The system of claim 1, wherein the cutting block portion further includes anchoring means for attaching the cutting block portion to bone and therewith fixing an orientation of the aperture therein when the cutting block portion is adjusted to a desired orientation.

4. The system of claim 3, wherein the aperture is in a form of a slot with opposing planer surfaces for guiding an oscillating bone cutting saw blade with respect to a cutting plane, and wherein fixing the orientation of the aperture sets the cutting block portion with respect to a desired cutting plane.

5. The system of claim 1, wherein each of the plurality of sensor units each have a common pre-defined shape that enables sensor unites to be interchangeable with respect to the cutting block first and second recess.

6. The system of claim 1, wherein the intermediate portion comprises at least one pivot between the attachment portion and the cutting block portion.

7. The system of claim 1, wherein the intermediate portion comprises at least one hinge between the attachment portion and the cutting block portion.

8. The system of claim 1, wherein the intermediate portion telescopically couples the attachment portion and the cutting block portion.

9. The system of claim 8, wherein the intermediate portion comprises at least one pivot between the attachment portion and the cutting block portion.

10. The system of claim 9, wherein the intermediate portion further comprises a hinge between the attachment portion and the cutting block portion and wherein the attachment portion and the cutting block portion are therewith adjustably coupled in three dimensions.

11. The system of claim 8, wherein the intermediate portion comprises a rack and pinion configured between the attachment portion and the cutting block portion for setting a vertical distance between the attachment portion and the cutting block portion.

12. The system of claim 8, wherein the intermediate portion comprises a hinge and a pivot between the attachment portion and the cutting block portion, the cutting block portion therewith adjustable vertically, rotationally about a vertical axis, and pivotally.

13. The system of claim 1, wherein at least one of the plurality of sensor units is a gyro sensor unit that communicates position information wirelessly to the client device.

14. The system of claim 1, wherein at least one of the plurality of sensor units is a magnetic sensor unit.

15. The system of claim 1, wherein the client device is configured to detect position information of each of the plurality of the sensors and construct a virtual model of a subject's skeletal anatomy based on kinematic data derived from position information from the plurality of sensors attached thereto.

16. The system of claim 15, wherein the client device is further configured to calculate at least one of a tibial and a femoral cutting plane based on individual kinematic data and muscular activity.

17. The system of claim 16, wherein the client device is further configured to display an interface screen depicting an avatar of the subject skeletal anatomy and superimpose the calculated cutting plane thereon.

18. The system of claim 17, wherein the client device is further configured to superimpose on the avatar an orientation of a guide cutting plane presented by the cutting block portion.

19. The system of claim 15, wherein the client device is further configured to calculate a thickness of the tibial cut.

20. The system of claim 15, wherein the client device is further configured to display a series of interface screens that queue users with respect to a resection workflow, including queues for placing position sensor units at predefined locations on a subject's ankle, hip, tibia, and femur.

21. The system of claim 20, wherein the queues further prompt users to attach the cutting block to at least one of the sensor units and adjust the cutting block portion relative to the attachment portion to achieve congruency with respect to a calculated cutting plane and a guide cutting plane presented by the cutting block portion.

22. The system of claim 21, wherein each of the calculated and guide cutting planes are presented as different colors until congruency is achieved.

23. The system of claim 21, wherein the client device further displays the avatar in a three-dimensional coordinate system and projects the cutting planes on at least one of the axial planes.
